(19) United States
(12) Patent Application Publication
(10) Pub. No.: US 2024/0366505 A1
(43) Pub. Date: Nov. 7, 2024

(54) Liquid Resin Extended-Release Oral Naltrexone Formulation for Treating Autism-Related Disorders

(71) Applicant: AARDVARK THERAPEUTICS, INC., San Diego, CA (US)

(72) Inventors: Tien-Li LEE, San Diego, CA (US); Zhenhuan ZHENG, San Diego, CA (US); Yu-Hsing TU, San Diego, CA (US)

(73) Assignee: AARDVARK THERAPEUTICS, INC., San Diego, CA (US)

(21) Appl. No.: 18/778,729

(22) Filed: Jul. 19, 2024

Related U.S. Application Data
(63) Continuation of application No. PCT/US2023/011649, filed on Jan. 26, 2023.
(60) Provisional application No. 63/303,285, filed on Jan. 26, 2022.

(51) Int. Cl. A61K 9/10; A61K 9/50; A61K 31/485; A61K 47/10; A61K 47/12; A61K 47/26; A61K 47/36

(52) U.S. Cl. CPC A61K 9/10; A61K 9/5026; A61K 9/5042; A61K 31/485; A61K 47/10; A61K 47/12; A61K 47/26; A61K 47/36

(57) ABSTRACT

There is disclosed a liquid resin extended-release oral naltrexone formulation suspension comprising from about 1.0 mg/ml to about 10.0 mg/ml naltrexone in a resin, wherein no more than 30% of the total naltrexone dose administered is released within one hour and wherein no more than 60% of the total naltrexone dose administered is released within two hours. Specifically, there is disclosed a method for treating a child with an autism-type disorder with approximately a teaspoon (about 5 ml) of a liquid resin extended-release oral naltrexone formulation suspension comprising from about 1.0 mg/ml to about 10.0 mg/ml naltrexone in a resin, wherein no more than 30% of the total naltrexone dose administered is released within one hour and wherein no more than 60% of the total naltrexone dose administered is released within two hours.

### CROSS-REFERENCE TO RELATED APPLICATIONS

This application is a continuation of International Patent Application No. PCT/US2023/011649, filed Jan. 26, 2023, which claims the benefit of priority of U.S. Provisional Application No. 63/303,285, filed on Jan. 26, 2022, the entire contents of each of which are incorporated herein by reference.

### TECHNICAL FIELD

The present disclosure provides a liquid resin extended-release oral naltrexone formulation suspension comprising from about 1.0 mg/mL to about 10.0 mg/mL naltrexone in a resin, wherein no more than 30% of the total dose administered is released within one hour and wherein no more than 60% of the total dose administered is released within two hours. Specifically, the present disclosure provides for a method for treating a child with an autism-type disorder with approximately a teaspoon (about 5 mL) of a liquid resin extended-release oral naltrexone formulation suspension comprising from about 1.0 mg/ml to about 10.0 mg/ml naltrexone in a resin, wherein no more than 30% of the total dose administered is released within one hour and wherein no more than 60% of the total dose administered is released within two hours. More specifically the present disclosure provides a liquid resin formulation that achieves a naltrexone formulation that releases no more than 30% of the total dose of naltrexone administered within one hour and no more than 60% of the total dose administered or naltrexone released within two hours comprising (a) a resinous core comprising naltrexone HCl and an ion exchange resin selected from the group consisting of polystyrene, a co-polymer of polystyrene and divinylbenzene; (b) a material coating the resinous core to form a particle, wherein the coating material is selected from the group consisting of cellulose acetate, cellulose diacetate, cellulose triacetate, cellulose acetate butyrate, cellulose acetate phthalate (CAP), cellulose acetate propionate, cellulose acetate phthalate (CAP), hypromellose phthalate (hydroxypropylmethylcellulose phthalate; HPMCP), polyvinyl acetate phthalate (PVAP); cellulose acetate trimellitate; polymethacrylates (e.g., Eudragit® L series, Eudragit® S series, Eudragit® FS, Kollicoat® MAE 100P, Acryl-EZE® 93A, Acryl-EZE® MP); hydroxypropylmethylcellulose acetate succinate (HPMC AS; Aqoat®); sodium alginate; alginic acid, shellac and combinations thereof, and (c) an aqueous thickened flavored and sweetened liquid for suspending the particles. Preferably, the liquid resin naltrexone formulation is administered qAM (upon waking).

### BACKGROUND

Autism spectrum disorder (ASD), a common pediatric health problem affecting 1 in 40 children in the United States, is a biologically based neurodevelopmental disorder characterized by persistent deficits in social communication and social interaction and restricted, repetitive patterns of behavior, interests, and activities, with symptoms generally observed in the first two years of life (Ozonoff et al. 2008, Autism Res, 1: 320-8). Autism is a complex developmental disability that interferes with, among other things, the normal development of the brain in the areas of social interaction and communication skills. It typically appears during the first three years of life and is the result of a neurological disorder which affects the functioning of the brain. Typically, autistic children and adults have difficulties in verbal and non-verbal communication, social interactions, and leisure or play activities.

In the past decades, a wide range of theories concerning the etiology of ASD has been proposed, including an opioid-excess theory. This theory was proposed by Panksepp in 1979 that autism would be an emotional disorder caused by excessive brain opioid activity (Panksepp, 1979, Trends in Neurosciences, 2: 174-77). In the brain, opioid activity depends on activation of endogenous opioid signaling through opioid receptors. Opioid receptors belong to the large family of G-protein-coupled receptors (GPCRs) and include three members: μ, δ and κ opioid receptors (Pellissier et al. 2018, Br. J. Pharmacol., 175: 2750-69). Endogenous opioid peptides, enkephalins, endorphins and dynorphins, are their preferential ligands, respectively, to promote opioid receptor activation. According to the main premise of the opioid-excess theory, peptides with opioid activity, derived from dietary sources, particularly containing gluten and casein, pass through an abnormally permeable intestinal membrane and enter the central nervous system (CNS). Such peptides exert neuroregulatory functions to impair balanced opioid receptor activity in the brain and cause excessive and/or prolonged opioid stimulation. This theory causes a deleterious impact on social behavior (Shattock and Whiteley 2002, Expert Opin. Ther. Targets, 6: 175-83.).

Exposure to high doses of opiates affects social behavior in the long term, as mice exposed to escalating doses of morphine or heroin displayed social interaction deficit up to 7 weeks after cessation of treatment (Pellissier et al. 2018, Br. J. Pharmacol., 175: 2750-69). As for the clinical findings, studies have reported elevated levels of endorphin fractions in the cerebral spinal fluid (CSF) of a group of children with autism and elevated β-endorphin levels in peripheral blood mononuclear cells (PBMCs) (Cazzullo et al. 1999, Eur. Neuropsychopharmacol., 9: 361-6; and Gillberg et al., 1985., Arch. Gen. Psychiatry, 42: 780-3). Evidence has also emerged for a possible familial connection. Leboyer et al. (Leboyer et al. 1992, J. Autism Dev. Disord., 22: 309-19) found an increase in the levels of β-endorphin protein immunoreactivity in over half of mothers of people with autism. Based on these findings, therefore, opioid antagonists like naltrexone should be beneficial to treat ASD.

A pediatric rare disease is Schaff-Yang Syndrome (SYS), a neurodevelopmental genetic disorder caused by truncating mutations in MAGEL2, located in the Prader-Willi critical region 15q11-15q13. SYS has clinical overlap with Prader Willi Syndrome (PWS) in the initial stages of life but becomes increasingly distinct throughout childhood and adolescence. Characteristics include intellectual disability/developmental delay and autism. Variation in phenotypic severity may depend on the specific location of the truncating mutation. The syndromes (i.e., SYS and PWS) share common features, primarily during infancy, including neonatal hypotonia, feeding difficulties/failure to thrive, respiratory distress, and DD/ID (developmental delay/intellectual disability). However, distinctions between the syndromes begin prenatally and increase throughout development. The earliest distinctive feature of SYS is the presence of joint contractures, which are frequently seen in SYS, while not typically described in the PWS phenotype. Individuals with SYS display more profound delays in motor and speech development and tend to manifest a more severe form of intellectual disability than those with PWS. There are no treatments for SYS.

Another related rare pediatric disorder is Bosch-Boonstra-Schaaf Optic Atrophy Syndrome (BBSOAS). It is extremely rare (less than 50 cases known) autosomal dominant disorder caused by pathogenic variants in the NR2F1 gene. Characteristics include delayed development/moderately impaired intellectual development, optic atrophy and autism. There are no treatments for BBSOAS.

There is no cure for autism and there are limited therapeutic options. Examples of medications used to treat symptoms associated with autism include: Serotonin re-uptake inhibitors (e.g., clomipramine, fluvoxamine, and fluoxetine) which have been used to treat depression, obsessive-compulsive behaviors, and anxiety. Stimulants, such as Ritalin, Adderall, and Dexedine, used to treat hyperactivity in children with ADHD, have also been used. They may increase focus, and decrease impulsivity and hyperactivity in autism, particularly in higher-functioning children. Unfortunately, adverse behavioral side effects are often observed. There are a wide variety of side effects are associated with such medications.

### Naltrexone (Immediate Release) and ASD

Since its original approval in 1984, the therapeutic effects of oral, immediate release naltrexone have been tested in pediatric patients in the clinic, including ASD. In a double-blind, placebo-controlled crossover study, 13 children (12 males and 1 female) with ASD, aged 3.4 to 8.3 years (mean 5.4 years), received a single daily immediate release oral dose of naltrexone at 1.0 mg/kg or placebo in a crossover fashion with a treatment duration of 12 or 14 days for each treatment phase (Kolmen et al. 1995, J. Am. Acad. Child Adolesc. Psychiatry, 34: 223-31). Changes in parent measures (Clinical Global Impressions (CGI), Conners Impulsivity-Hyperactivity Factor (IHF), and Side-Effects SE-Restlessness) and Teacher CGI achieved statistical significance. In another double-blind placebo-controlled crossover study involving 23 ASD children (aged 3-7 years), all subjects received 20 mg naltrexone immediate release per day (except one boy on an adjusted dosage of 40 mg per day) for 4 weeks and 20 completed final analyses (Willemsen-Swinkels et al., 1996, Biol. Psychiatry, 39: 1023-31). Naltrexone treatment brought about significant changes in the behavior ratings completed by the teachers (Aberrant Behavior Checklist (ABC) scores). Significant difference was also achieved in the CGI ratings of the teachers. High dose immediate release oral naltrexone (0.5, 1.0 and 2.0 mg/kg) has been shown to reverse autistic behavior and gaze aversion (Lensing et al., Neuropsychobiology. 1995; 31: 16-23). And a reduction in opioid tone in ASD patents correlated with treatment response (Cazullo et al., Eur. Neuropsychopharmacol. 1999; 9: 361-6).

Major side effects include trouble sleeping, anxiety, nausea, and headaches. In those still on opioids or alcohol, opioid withdrawal may occur. Use is not recommended in people with liver failure. It is unclear if use is safe during pregnancy. These adverse effects are analogous to the symptoms of opioid withdrawal. The side effects of naltrexone by incidence are as follows:
Greater than 10%: difficulty sleeping, anxiety, nervousness, abdominal pain/cramps, nausea and/or vomiting, low energy, joint/muscle pain, and headache.
Less than 10%: loss of appetite, diarrhea, constipation, thirstiness, increased energy, feeling down, irritability, dizziness, skin rash, delayed ejaculation, erectile dysfunction, and chills.

Therefore, there is a need to better administer naltrexone to lessen Cmax peaks, so as to mitigate side effects or lessen their impact.

### Pharmacokinetics and Metabolism in Humans

The clinical pharmacokinetic profile of naltrexone immediate-release tablets (50 mg) is known and summarized in the REVIA® Label and in a National Institute on Drug Abuse Research Monograph (NIDA Research Monograph 28, 1981). Naltrexone is well absorbed orally and subject to significant first pass metabolism with oral bioavailability estimates ranging from 5 to 40%. The activity of naltrexone is believed to be due to both parent and the 6-β-naltrexol metabolites. Both parent drug and metabolites are excreted primarily by the kidney (53% to 79% of the dose). Urinary excretion of unchanged naltrexone accounts for less than 2% of an oral dose based on immediate release formulations. Fecal excretion is a minor elimination pathway. The mean elimination half-life (ti/2) values for immediate release oral naltrexone and 6-β-naltrexol are 4 hours and 13 hours, respectively. Naltrexone and 6-β-naltrexol are dose-proportional in terms of AUC and Cₘₐₓ over the range of 50-200 mg and do not accumulate after 100 mg daily doses.

According to the data reported in ANDA #075434, following a single administration of 50 mg REVIA® tablets to 36 healthy adult male volunteers, the Cₘₐₓ for REVIA® and its major metabolite, 6-β-naltrexol were 7.3 ng/mL (62.5%) and 85.6 ng/ml (33.4%), and AUC_{(0-inf)} were 23.1 ng*h/mL (55.1%) and 717.3 ng*h/mL (21.8%), respectively, suggesting that the systemic exposure of 6-β-naltrexol is over 10-fold higher relative to naltrexone. Multiple dose PK of naltrexone Immediate-release tablets (50 mg REVIA® tablets administered once daily for 7 days in 24 healthy adult volunteers; (Mason et al. 2002, Neuropsychopharmacology, 27: 596-606) was Cₘₐₓ for REVIA® and its major metabolite, 6-β-naltrexol were 11.8±6.55 ng/mL and 96.1±21.05 ng/mL), and AUC_{(0-τ)} were 38.6 16.53 ng*h/mL and 788±134.8 ng*h/mL, respectively. Therefore, following oral administration, naltrexone undergoes rapid and nearly complete absorption with approximately 96% of the dose absorbed from the gastrointestinal tract. Peak plasma levels of both naltrexone and 6-β-naltrexol occur within 1 hour of dosing. According to the data reported in ANDA #075434, following the single administration of 50 mg REVIA® tablets to 36 healthy adult male volunteers: Mean (CV %) of Tₘₐₓ for naltrexone: 1.07 hours (27.7%); Mean (CV %) of Tₘₐₓ for 6-β-naltrexol: 1.05 hours (27.9%).

In a placebo-controlled clinical trial, treatment with 50 mg naltrexone once daily produced significantly more adverse effects (Bonferroni-adjusted p=0.003, p=0.049, and p=0.004 for weeks 1, 5, and 8, respectively), resulting in poorer medication compliance (˜50% vs. 70% for naltrexone vs. placebo at the end of Week 11 of the randomization treatment period) and a greater rate of early discontinuation of treatment than placebo (Kranzler et al. Neuropsychopharmacology. 2000; 22(5):493-503). Further, the data reported in ANDA #075434, provides the PK parameters, including Cₘₐₓ, of naltrexone at 50 mg instant release tablets.

Without being bound by theory, it may be the case the adverse effects of naltrexone derive from higher systemic exposure, particularly Cₘₐₓ. A study tested tolerance of a sustained-release naltrexone subcutaneous injection formulation in alcohol-dependent subjects. After injection of the sustained-release formulation, naltrexone plasma concentrations exceeded a mean of 1 ng/mL for 21 days (range 1-6 ng/mL), which was lower than the plasma Cₘₐₓ concentrations with administration of naltrexone 50 mg instant release tablets (11.8 ng/mL). Although the frequency of adverse effects produced under this PK profile by the sustained-release formulation was higher as compared to placebo, there was no statistically significant difference. And no complaints of a severe nature were reported during the injection period. All adverse effects resolved spontaneously (Kranzler et al. Alcohol Clin. Exp. Res. 1998; 22(5):1074-9).

Accordingly, given the side effects of naltrexone treatments, there is a need in the art to provide a naltrexone formulation for autistic children/patients that (1) can be administered to such patients; and (2) mitigates the side effects associated with naltrexone treatments. The present disclosure provides a naltrexone formulation that addresses both of the forgoing needs. Therefore, there is a need in the art for an extended-release oral formulation that dampens naltrexone's exposure profile and mitigates undesirable acute side effects associated with oral administration of immediate release naltrexone formulations.

### SUMMARY

The present disclosure addresses the problem how to utilize a new and improved pharmacokinetic formulation of naltrexone to mitigate naltrexone side effects, particularly in a liquid formulation so it can be administered to children, particularly to treat various autism spectrum indications. Specifically, the present disclosure provides a liquid resin extended-release oral naltrexone formulation suspension comprising from about 1.0 mg/ml to about 10.0 mg/ml naltrexone in a resin, wherein no more than 30% of the total naltrexone dose administered is released within one hour and wherein no more than 60% of the total naltrexone dose administered is released within two hours. Specifically the present disclosure provides a liquid resin formulation that achieves a naltrexone formulation that releases no more than 30% of the total dose of naltrexone administered within one hour and no more than 60% of the total dose administered of naltrexone released within two hours, comprising (a) a resinous core comprising naltrexone HCl and an ion exchange resin selected from the group consisting of polystyrene, a co-polymer of polystyrene, divinylbenzene; a chelating agent and a granulating agent (b) a material coating the resinous core to form a particle comprising a cellulose acetate-containing polymer and a plasticizer, selected from the group consisting of cellulose acetate, cellulose diacetate, cellulose triacetate, cellulose acetate butyrate, cellulose acetate phthalate (CAP), cellulose acetate propionate, cellulose acetate phthalate (CAP), hypromellose phthalate (hydroxypropylmethylcellulose phthalate; HPMCP), polyvinyl acetate phthalate (PVAP); cellulose acetate trimellitate; polymethacrylates (e.g., Eudragit® L series, Eudragit® S series, Eudragit® FS, Kollicoat® MAE 100P, Acryl-EZE® 93A, Acryl-EZE® MP); hydroxypropylmethylcellulose acetate succinate (HPMC AS; Aqoat®); sodium alginate; alginic acid, shellac and combinations thereof, (c) an aqueous thickened flavored and sweetened liquid for suspending the particles, and optionally (d) a preservative.

The present disclosure further provides for a method for treating a child with an autism-type disorder with approximately a teaspoon (about 5 ml) of a liquid resin extended-release oral naltrexone formulation suspension comprising from about 1.0 mg/ml to about 10.0 mg/ml naltrexone in a resin, wherein no more than 30% of the total naltrexone dose administered is released within one hour and wherein no more than 60% of the total naltrexone dose administered is released within two hours. Preferably, the liquid resin formulation comprises (a) a resinous core comprising naltrexone HCl and an ion exchange resin selected from the group consisting of polystyrene, a co-polymer of polystyrene and divinylbenzene; (b) a material coating the resinous core to form a particle, wherein the coating material is selected from the group consisting of cellulose acetate, cellulose diacetate, cellulose triacetate, cellulose acetate butyrate, cellulose acetate phthalate (CAP), cellulose acetate propionate, cellulose acetate phthalate (CAP), hypromellose phthalate (hydroxypropylmethylcellulose phthalate; HPMCP), polyvinyl acetate phthalate (PVAP); cellulose acetate trimellitate; polymethacrylates (e.g., Eudragit® L series, Eudragit® S series, Eudragit® FS, Kollicoat® MAE 100P, Acryl-EZE® 93A, Acryl-EZE® MP); hydroxypropylmethylcellulose acetate succinate (HPMC AS; Aqoat®); sodium alginate; alginic acid, shellac and combinations thereof, and (c) an aqueous thickened flavored and sweetened liquid for suspending the particles. Preferably, the liquid resin naltrexone formulation is administered qAM (upon waking).

The present disclosure further provides for a method for treating a child with a rare pediatric disease Schaff-Yang Syndrome (SYS) or Bosch-Boonstra-Schaaf Optic Atrophy Syndrome (BBSOAS) with approximately a teaspoon (about 5 ml) of a liquid resin extended-release oral naltrexone formulation suspension comprising from about 1.0 mg/ml to about 10.0 mg/ml naltrexone in a resin, wherein no more than 30% of the total naltrexone dose administered is released within one hour and wherein no more than 60% of the total naltrexone dose administered is released within two hours. Preferably, the liquid resin formulation comprises (a) a resinous core comprising naltrexone HCl and an ion exchange resin selected from the group consisting of polystyrene, a co-polymer of polystyrene and divinylbenzene; (b) a material coating the resinous core to form a particle, wherein the coating material is selected from the group consisting of cellulose acetate, cellulose diacetate, cellulose triacetate, cellulose acetate butyrate, cellulose acetate phthalate (CAP), cellulose acetate propionate, cellulose acetate phthalate (CAP), hypromellose phthalate (hydroxypropylmethylcellulose phthalate; HPMCP), polyvinyl acetate phthalate (PVAP); cellulose acetate trimellitate; polymethacrylates (e.g., Eudragit® L series, Eudragit® S series, Eudragit® FS, Kollicoat® MAE 100P, Acryl-EZE® 93A, Acryl-EZE® MP); hydroxypropylmethylcellulose acetate succinate (HPMC AS; Aqoat®); sodium alginate; alginic acid, shellac and combinations thereof, and (c) an aqueous thickened flavored and sweetened liquid for suspending the particles. Preferably, the liquid resin naltrexone formulation is administered qAM (upon waking).

### BRIEF DESCRIPTION OF THE FIGURES

FIG. 1 shows release kinetics of naltrexone (50 mg) for immediate release (IR) and extended release (ER), wherein the ER formulation is described in Example 1 herein.

FIG. 2 shows a modeled and observed first order release model pharmacokinetics for naltrexone immediate release 50 mg tablets from generic ANDA filings. The first order release model was calculated as: dA/dt=−A*kr and A(0)=100% @ t=0.

FIGS. 3A and 3B show simulated pharmacokinetic curves at Day 1 for single dose oral IR and disclosed ER formulations of naltrexone and the first pass liver metabolite 6-β-naltrexol.

FIGS. 4A and 4B show simulated pharmacokinetic curves at Day 10 for once daily dosing for oral IR and disclosed ER formulations of naltrexone and the first pass liver metabolite 6-β-naltrexol.

FIG. 5 shows a drug release curve for immediate release naltrexone and a disclosed extended-release liquid oral suspension extended release.

FIG. 6 shows drug release profiles for three liquid naltrexone extended-release formulations having different particle coatings. In the Figure, CAB is cellulose acetate butyrate coated particle formulation of Table 3, CAB and HPMCP (hypomellose phthalate) formulation of Table 6, and EC (ethyl cellulose) formulation of Table 7. The EC formulation appeared to satisfy the present criteria of no more than 25% of the total dose of naltrexone administered within one hour and no more than 60% of the total dose administered or naltrexone released within two hours.

FIG. 7 shows the pharmacokinetic profile of oral naltrexone as described in Tables 18 and 19 in Example 8.

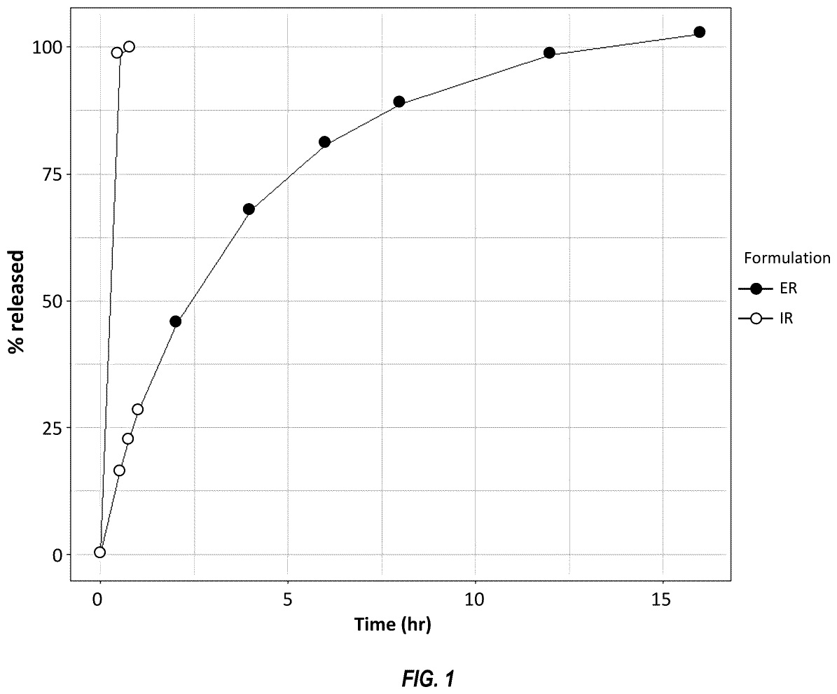

### DETAILED DESCRIPTION

### Pharmacokinetics

The method for treating autism spectrum disorders is to dose qAM (once per day in the morning), as early upon awakening as possible. Naltrexone only has a half-life of about 4 hours, but the main metabolite, 6-beta-naltrexol, has activity as an opioid antagonist too and has a half-life of up to 13 hours. The reasons for the qAM dosing schedule, study duration, and targeted formulation characteristics are multi-fold:

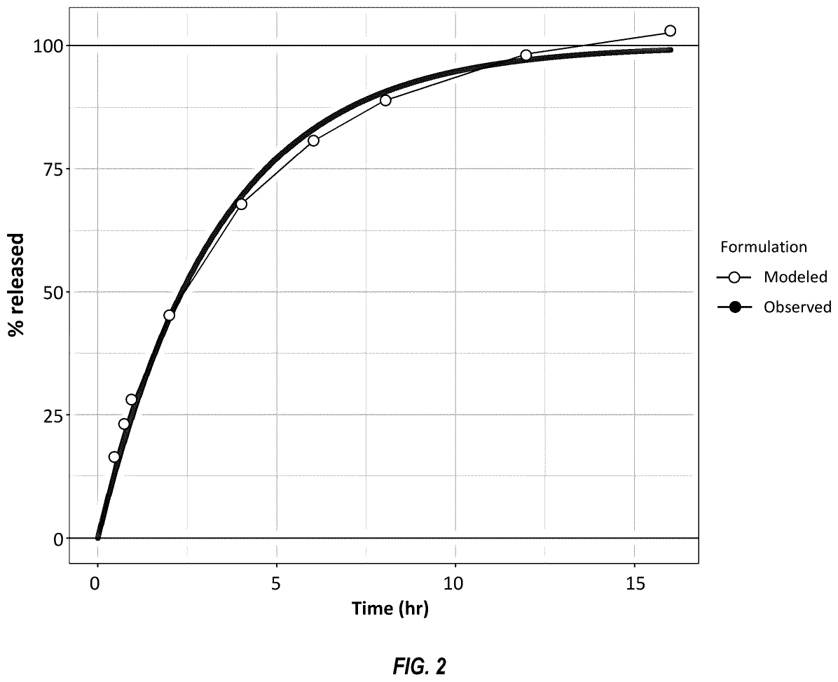

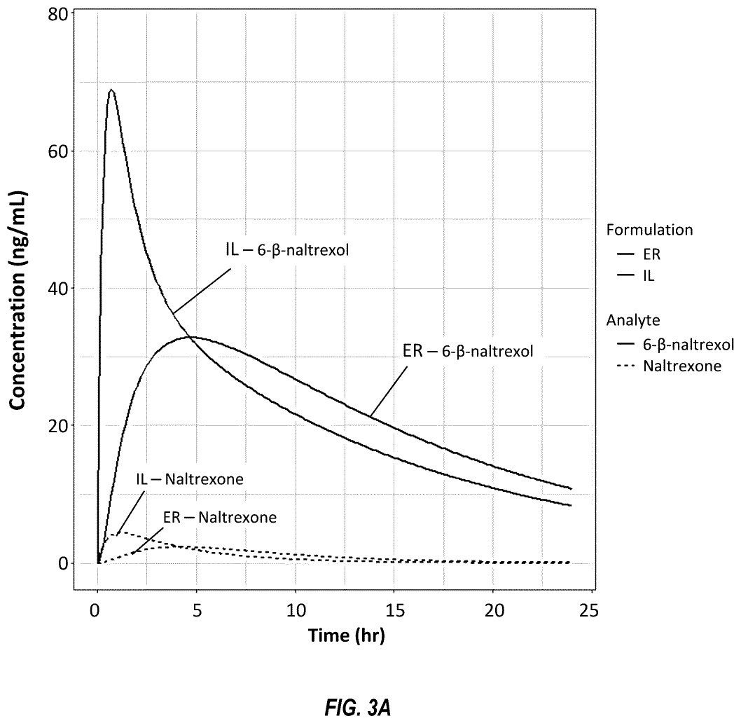

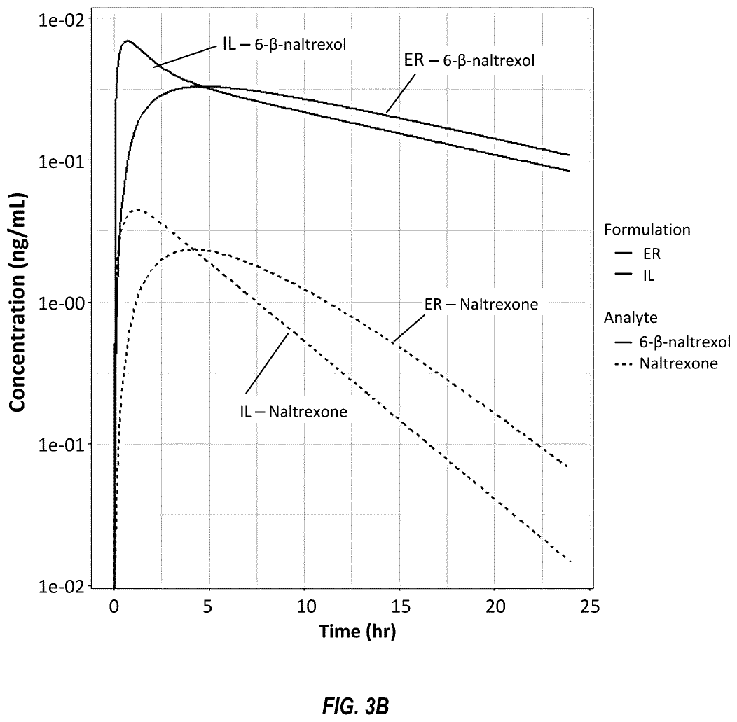

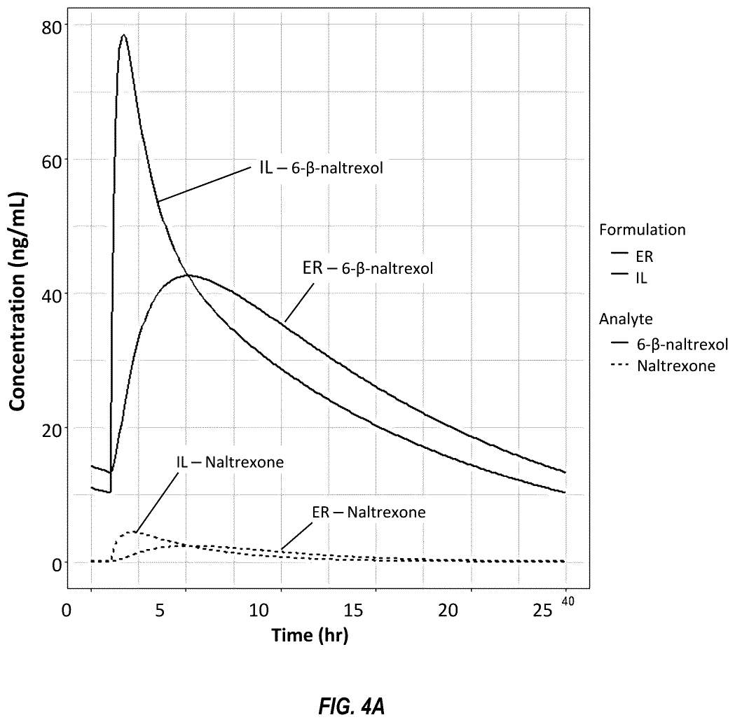

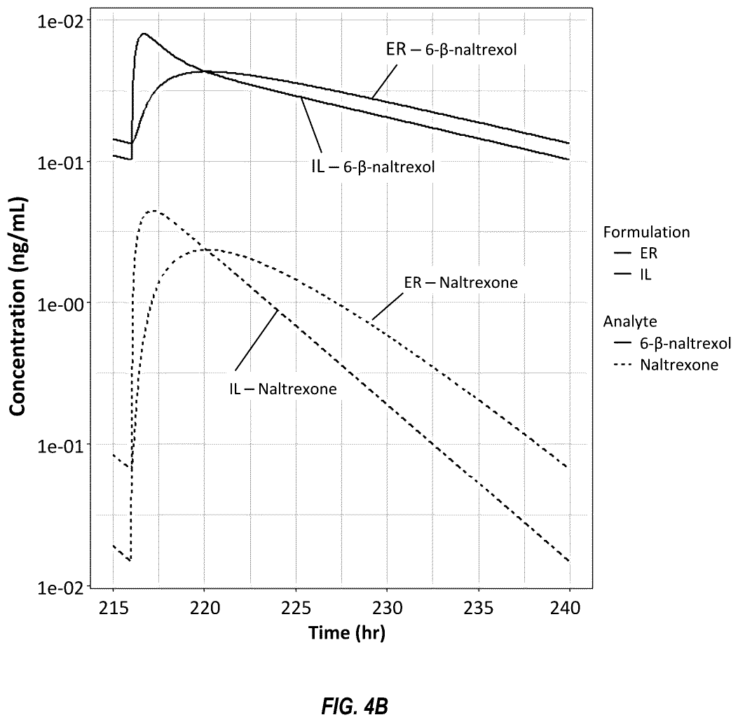

1. Dose early in the morning to allow drug effect to peak in daytime when behavior issues are most important. But allow drug effect to wane before bedtime so as to not interfere with sleep.
2. The current approved naltrexone tablet formulation has an onset of 15 min and peak plasma concentration in 1 hr. Therefore, the current liquid resin formulation and the pharmacokinetic modeling provided herein shows that in a clinical trial, the present formulation will help reduce side effects (nausea, hyperalgesia, dizziness). Opioid agonists have the greatest effects on euphoria and analgesia when the “slope” of serum exposure is the steepest, that is, injecting opioids has a more dramatic effect than the same dose equivalent given orally with slower absorption. Similarly, naltrexone a steep “slope” for exposure also elicits the most side effects. As the PK modeling shows, the naltrexone release characteristics of the disclosed liquid resin formulation provides better slope and Cmax characteristics to significantly alleviate side effects yet provide substantially the same AUC (area under the curve) as the dose-equivalent IR (immediate release) commercially approved formulation. Yet the disclosed ER (extended release) formulation shows a lower Cmax and lesser slope of the concentration/time curve. (FIGS. 2 and 3). This type of drug profile also slows down and blunts any tolerance that might develop against the treatment as is shown in the model for day 10 in FIG. 4.

The present disclosure further provides a liquid resin extended release oral naltrexone liquid suspension formulation comprising from about 1 mg/ml to about 10 mg/ml in an ion exchange resin and coated with an extended release rate polymer to administer a teaspoon (about 5 ml) to a child having autism across a spectrum of autism symptoms, wherein the naltrexone is within a coated naltrexone-anion exchange resin complex, wherein the resin complex is in a matrix with a water insoluble polymer or copolymer or hydrophilic polymer, comprising: (a) naltrexone bound to a pharmaceutically acceptable, water insoluble, anion exchange resin to form a naltrexone-ion exchange resin coated particle; wherein the naltrexone comprises about 40% by weight to about 75% by weight of the naltrexone-ion exchange resin complex-matrix. Preferably, the ion exchange resin is a copolymer comprising styrene and divinylbenzene comprising quaternary ammonium functional groups. Preferably, the anion exchange resin is a cholestyramine resin. Preferably, the modified release barrier coating comprises a water-permeable, water insoluble polymer and a plasticizer. More preferably, he water-permeable, water-insoluble polymer in the barrier coating comprises polyvinyl acetate. Preferably, the matrix comprises the naltrexone-anion exchange resin complex and the hydrophilic polymer. More preferably, the hydrophilic polymer in the matrix comprises a polyvinylpyrrolidone. Preferably, the matrix comprises the particulate drug-anion exchange resin complex, polyvinylpyrrolidone and a surfactant.

The present disclosure further provides the following embodiments of Coated Granule based on the pharmacokinetic parameters achieved in Example 8 here:

Coated Granule 1, wherein the coated drug-ion exchange resin complex granule provides a pharmacokinetic profile, having one or more of the following characteristics a-e:
a) Tₘₐₓ of about 5 to about 7 hours, for example about 5 hours, about 6 hours or about 7 hours;
b) T_{1/2} of about 5 to about 12 hours, for example about 5 to about 11 hours; about 6 hours, about 7 hours, about 8 hours, about 9 hours, about 10 hours or about 11 hours;
c) Cₘₐₓ of about 1.5 to about 4 hours, for example about 1.5 hours, about 2 hours about 2.5 hours, about 3 hours, about 3.5 hours or about 4 hours;
d) AUCₜ of about 26 to about 32 hr·ng/mL, for example about 26, about 27, about 28, about 29, about 30, about 31 or about 32 hr·ng/mL; and
e) AUC of about 28 to about 35 hr·ng/mL, for example about 28, about 29, about 30, about 31, about 32, about 33, about 34 of about 35 hr·ng/mL.

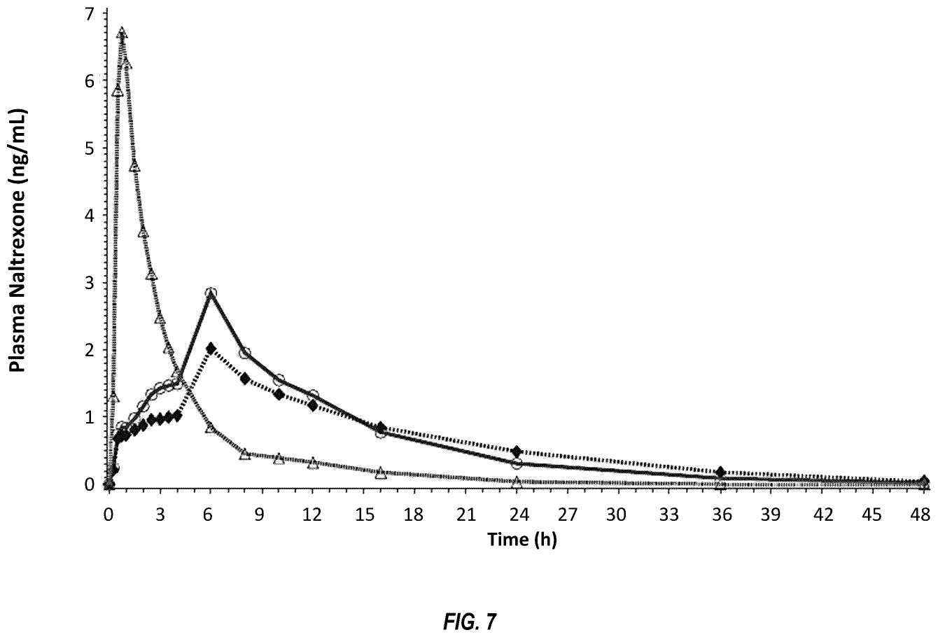

Coated Granule 1.1, wherein the coated drug-ion exchange resin complex granule has a pharmacokinetic profile having two or more of the characteristics a-e, or three or more of the characteristics a-e, or four or more of the characteristics a-e, or all five of the characteristics a-e. More specifically, wherein the coated drug-ion exchange resin complex granule has a pharmacokinetic profile is substantially in accordance with Formulation 1 or Formulation 2 in Example 8 or FIG. 7. Alternatively, wherein the Mean Plasma Naltrexone Concentration (ng/ml) of the patient over the period 2.5 hours post administration to 4 hours post administration increases by least 4%; or from about 4% to about 20%; or from about 4% to about 15%; or from about 4% to about 12%, or from about 5% to about 12%, or by about 4%, about 5%, about 6%, about 7%, about 8%, about 9%, about 10%, about 11% or about 12%.

Any of the preceding Coated Granules, wherein the Mean Plasma Naltrexone Concentration (ng/ml) of the patient over the period 4 hours post administration to 6 hours post administration increases by least about 80%; or from about 80% to about 120%; or from about 80% to about 110%; or from about 85% to about 105%, or about 86%, about 87%, about 88%, about 89%, about 90%, about 91%, about 92%, about 93%, about 94%, about 95%, about 96%, about 97%, about 98%, about 99%, about 100%, about 101%, about 102%, about 103%, about 104% or about 105%.

Any of the preceding Coated Granules, wherein the Mean Plasma Naltrexone Concentration (ng/ml) of the patient over the period 6 hours post administration to 8 hours post administration decreases by no more than about 40%; or from about 15% to about 40%; or from about 20% to about 35%; or about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%.

Any of the preceding Coated Granules, wherein the ratio of the Mean Plasma Naltrexone Concentration (Cₜ; ng/ml) to the Maximum Plasma Naltrexone Concentration (Cₘₐₓ; ng/ml) of the patient three hours after administration is from about 0.4 to about 0.6; for example, from about 0.4 to about 0.55, for example from about 0.45 to about 0.5.

Any of the preceding Coated Granules, wherein the ratio of the Mean Plasma Naltrexone Concentration (Cₜ; ng/ml) to the Maximum Plasma Naltrexone Concentration (Cₘₐₓ; ng/ml) of the patient six hours after administration is from about 0.8 to about 1.2; for example, from about 0.9 to about 1.1, for example from about 0.95 to about 1.05.

Any of the preceding Coated Granules, wherein the ratio of the Mean Plasma Naltrexone Concentration (Cₜ; ng/ml) to the Maximum Plasma Naltrexone Concentration (Cₘₐₓ; ng/ml) of the patient eight hours after administration is from about 0.5 to about 0.9; for example, from about 0.6 to about 0.8, for example from about 0.65 to about 0.8.

Any of the preceding Coated Granules, wherein the ratio of the Mean Plasma Naltrexone Concentration (Cₜ; ng/ml) to the Maximum Plasma Naltrexone Concentration (Cₘₐₓ; ng/ml) of the patient ten hours after administration is from about 0.4 to about 0.8; for example, from about 0.5 to about 0.7.

Any of the preceding Coated Granules, wherein the ratio of the Mean Plasma Naltrexone Concentration (Cₜ; ng/ml) to the Maximum Plasma Naltrexone Concentration (Cₘₐₓ; ng/ml) of the patient twelve hours after administration is from about 0.3 to about 0.7; for example, from about 0.4 to about 0.6.

Any of the preceding Coated Granules, wherein the ratio of the Mean Plasma Naltrexone Concentration (Cₜ; ng/ml) to the Maximum Plasma Naltrexone Concentration (Cₘₐₓ; ng/ml) of the patient is one or more of a-e below:
a) three hours after administration, from about 0.4 to about 0.6; for example, from about 0.4 to about 0.55, for example from about 0.45 to about 0.5;
b) six hours after administration, from about 0.8 to about 1.2; for example, from about 0.9 to about 1.1, for example from about 0.95 to about 1.05;
c) eight hours after administration, from about 0.5 to about 0.9; for example, from about 0.6 to about 0.8, for example from about 0.65 to about 0.8;
d) ten hours after administration, from about 0.4 to about 0.8; for example, from about 0.5 to about 0.7; and
e) twelve hours after administration, from about 0.3 to about 0.7; for example, from about 0.4 to about 0.6.

Alternatively, wherein the ratio of the Mean Plasma Naltrexone Concentration (Cₜ; ng/ml) to the Maximum Plasma Naltrexone Concentration (Cₘₐₓ; ng/ml) of the patient is
a) three hours after administration, from about 0.4 to about 0.6; for example, from about 0.4 to about 0.55, for example from about 0.45 to about 0.5; and
b) six hours after administration, from about 0.8 to about 1.2; for example, from about 0.9 to about 1.1, for example from about 0.95 to about 1.05.

Alternatively, wherein the ratio of the Mean Plasma Naltrexone Concentration (Cₜ; ng/ml) to the Maximum Plasma Naltrexone Concentration (Cₘₐₓ; ng/ml) of the patient is
b) six hours after administration, from about 0.8 to about 1.2; for example, from about 0.9 to about 1.1, for example from about 0.95 to about 1.05; and
c) eight hours after administration, from about 0.5 to about 0.9; for example, from about 0.6 to about 0.8, for example from about 0.65 to about 0.8.

Alternatively, wherein the ratio of the Mean Plasma Naltrexone Concentration (Cₜ; ng/ml) to the Maximum Plasma Naltrexone Concentration (Cₘₐₓ; ng/ml) of the patient is a) three hours after administration, from about 0.4 to about 0.6; for example, from about 0.4 to about 0.55, for example from about 0.45 to about 0.5; and
b) six hours after administration, from about 0.8 to about 1.2; for example, from about 0.9 to about 1.1, for example from about 0.95 to about 1.05; and
c) eight hours after administration, from about 0.5 to about 0.9; for example, from about 0.6 to about 0.8, for example from about 0.65 to about 0.8; and
d) ten hours after administration, from about 0.4 to about 0.8; for example, from about 0.5 to about 0.7.

Preferably the ion exchange resin comprises a polystyrene polymer, or a co-polymer of polystyrene and divinylbenzene, or a matrix of sulfonated polymers composed of polystyrene cross-linked with about 8% of divinylbenzene. Preferably, the ion exchange resin has an ion-exchange capacity of about 4.5 to 5.5 meq/g of dry resin. And wherein the w/w ratio of the ion exchange resin to the Naltrexone or pharmaceutically acceptable salt thereof present in the Coated Granules is about 5:1 to 1:5, e.g., about 3:1 to 1:3, e.g., about 2:1 to 1:2, e.g., about 2:1.

Preferably, the cellulose acetate-containing polymer is selected from the group consisting of cellulose acetate, cellulose diacetate, cellulose triacetate, cellulose acetate butyrate, cellulose acetate phthalate, cellulose acetate propionate, and combinations thereof. More preferably, the cellulose acetate-containing polymer is selected from cellulose acetate, cellulose acetate butyrate, and combinations thereof. Most preferably, the cellulose acetate-containing polymer comprises cellulose acetate butyrate.

Preferably, the coated drug-ion exchange resin complex granule further comprises an enteric polymer; wherein the enteric polymer is selected from the group consisting of a phthalate-containing polymer; cellulose acetate trimellitate; polymethacrylates; hydroxypropylmethylcellulose acetate succinate (HPMC AS; Aqoat®); sodium alginate; alginic acid; shellac and combinations thereof.

Preferably, the coated drug-ion exchange resin complex granule further comprises an anti-tacking agent; for example and not limited to talc, glycerol monostearate, magnesium stearate or kaolin.

Preferably, the coated drug-ion exchange resin complex granule further comprises a granulating agent selected from benzophenone, butyl phthalyl butyl glycollate, camphor, alpha-cresyl-p-toluene sulfonate, cyclohexyl-p-toluene sulfonamide, diamyl phthalate, dibutyl phthalate, dibutyl sebacate, dibutyl succinate, dibutyl tartrate, diethoxyethyl adipate, diethoxyethyl phthalate, diethyl adipate, diethylene glycol dipropionate, diethyl phthalate, diethyl sebacate, diethyl succinate, diethyl tartrate, dimethoxyethyl adipate, dimethoxyethyl phthalate, dimethyl phthalate, dipropyl phthalate, ethyl benzoyl benzoate, ethylene glycol diacetate, ethylene glycol dibutyrate, ethylene glycol dipropionate, ethyl phthalyl ethyl glycolate, methyl benzoyl benzoate, methyl phthalyl ethyl glycolate, o- or p-toluene ethyl sulfonamide, triacetin, tributyl citrate, tributyl phosphate, tributyrin, tricresyl phosphate, triethylene glycol diacetate, triethylene glycol dibutyrate, triethylene glycol diproprionate, triphenyl phosphate, tripropionin, trimellitates (e.g., tri-(2-ethylhexyl)trimellitate, tri-(isononyl)trimellitate, tri-(isodecyl)trimellitate, tri-(isotridecyl)trimellitate), adipates (e.g., bis(2-ethylhexyl)adipate), sebacates (e.g., dibutyl sebacate, di-(2-ethylhexyl)sebacate), glycerol triacetate, alkyl citrates (triethyl citrate, acetyl triethyl citrate, tributyl citrate, acetyl trybutyl citrate, tri(2-ethylhexyl)citrate, acetyl trioctyl citrate, trihexyl citrate, butryyl trihexyl citrate), vegetable oils, epoxidized soybean oil, epoxidized linoleic oil, azelates, dibenzoates, terephthalates, 1,2-cyclohexane dicarboxylic acid diisononyl ester, alkyl sulphonic acid phenyl ester, organophosphates (e.g., tricresyl (methyl phenyl) phosphate, 2-ethylhexyldiphenyl phosphate), glycols (e.g., propylene glycol, polyethylene glycol, triethylene glycol di-2hexanoate) triacetin, triethyl citrate, dibutyl sebacate, vegetable oil, lipids and combinations thereof. Preferably, the granulating agent is polyethylene glycol.

Preferably, the plasticizer in the coating is selected from benzophenone, butyl phthalyl butyl glycollate, camphor, alpha-cresyl-p-toluene sulfonate, cyclohexyl-p-toluene sulfonamide, diamyl phthalate, dibutyl phthalate, dibutyl sebacate, dibutyl succinate, dibutyl tartrate, diethoxyethyl adipate, diethoxyethyl phthalate, diethyl adipate, diethylene glycol dipropionate, diethyl phthalate, diethyl sebacate, diethyl succinate, diethyl tartrate, dimethoxyethyl adipate, dimethoxyethyl phthalate, dimethyl phthalate, dipropyl phthalate, ethyl benzoyl benzoate, ethylene glycol diacetate, ethylene glycol dibutyrate, ethylene glycol dipropionate, ethyl phthalyl ethyl glycolate, methyl benzoyl benzoate, methyl phthalyl ethyl glycolate, o- or p-toluene ethyl sulfonamide, triacetin, tributyl citrate, tributyl phosphate, tributyrin, tricresyl phosphate, triethylene glycol diacetate, triethylene glycol dibutyrate, triethylene glycol diproprionate, triphenyl phosphate, tripropionin, trimellitates (e.g., tri-(2-ethylhexyl)trimellitate, tri-(isononyl)trimellitate, tri-(isodecyl)trimellitate, tri-(isotridecyl)trimellitate), adipates (e.g., bis(2-ethylhexyl)adipate), sebacates (e.g., dibutyl sebacate, di-(2-ethylhexyl)sebacate), glycerol triacetate, alkyl citrates (triethyl citrate, acetyl triethyl citrate, tributyl citrate, acetyl trybutyl citrate, tri(2-ethylhexyl)citrate, acetyl trioctyl citrate, trihexyl citrate, butryyl trihexyl citrate), vegetable oils, epoxidized soybean oil, epoxidized linoleic oil, azelates, dibenzoates, terephthalates, 1,2-cyclohexane dicarboxylic acid diisononyl ester, alkyl sulphonic acid phenyl ester, organophosphates (e.g., tricresyl (methyl phenyl) phosphate, 2-ethylhexyldiphenyl phosphate), glycols (e.g., propylene glycol, polyethylene glycol, triethylene glycol di-2hexanoate) triacetin, triethyl citrate, dibutyl sebacate, vegetable oil, lipids and combinations thereof. Preferably, the plasticizer comprises about 2.5 to about 25% w/w, or about 5 to about 15% w/w, or about 8 to about 12% w/w, or about 10% w/w of the solids in the coating.

### Naltrexone Particles

The particles are coated naltrexone-ion exchange resin (drug resin complex) having a resinous core surrounded by a polymer coating. A naltrexone resin complex is formed by mixing a naltrexone HCl and a resin (e.g., a polystyrene sulfonate resin) to facilitate protonation of naltrexone HCl (salt form or free base form) and subsequent bonding (e.g., ionic bonding) between the resinous polymer and naltrexone. This reaction may be carried out in an excess of drug until the drug no longer binds to the resinous polymer. The drug resin complex is collected by filtration and washed with appropriate solvents to remove any unbound drug or by-products. The complexes can be air-dried in trays, in a fluid bed dryer, or other suitable dryer, at room temperature or at elevated temperature.

In various embodiments, the amount of drug that can be loaded onto a resin ranges from about 1% to about 75% by weight of the drug-ion exchange resin particles. In some embodiments, drug loads of about 10% to about 40% by weight, e.g., about 15% to about 30% by weight, of the drug ion exchange resin particles may be achieved.

The polymer coating comprises a combination of a cellulose acetate-containing polymer, alone or in combination with one or more enteric polymers. As used herein, the term “cellulose acetate-containing polymer” means a polymer comprising at least one mono-, di- or tri-acetylated cellulose (i.e., β(1→4) linked glucose) monomer. The cellulose acetate-containing polymer is selected from the group consisting of cellulose acetate, cellulose diacetate, cellulose triacetate, cellulose acetate butyrate, cellulose acetate phthalate, cellulose acetate propionate, and combinations thereof. Preferably, the cellulose acetate polymer is cellulose acetate or cellulose acetate butyrate.

Optionally, the cellulose acetate polymer is mixed with a phthalate-containing polymer to facilitate a more uniform coating of the naltrexone-ion exchange resin complex and/or enhances the tensile strength of the barrier coating, wherein the phthalate-containing polymer is selected from the group consisting of a phthalate-containing polymer; cellulose acetate trimellitate; polymethacrylates; hydroxypropylmethylcellulose acetate succinate (HPMC AS; Aqoat®); sodium alginate; alginic acid; shellac, monomers of dimethyl phthalate, diethyl phthalate, diallyl phthalate, di-n-propyl phthalate, di-n-butyl phthalate, diisobutyl phthalate, butyl cyclohexyl phthalate, di-n-pentyl phthalate, dicylcohexyl phthalate, butyl benzyl phthalate, di-n-hexyl phthalate, diisohexyl phthalate, diisoheptyl phthalate, butyl decyl phthalate, dibutoxy ethyl phthalate, di(2-ethylhexyl) phthalate, di(n-octyl) phthalate, diisooctyl phthalate, n-octyl n-decyl phthalate, diisononyl phthalate, di(2-propylheptyl) phthalate, diisodecyl phthalate, diundecyl phthalate, diisoundecyl phthalate, ditridecyl phthalate, diisotridecyl phthalate, polyethylene terephthalate, cellulose phthalate, cellulose acetate phthalate (CAP), hydroxypropylmethylcellulose phthalate (HPMCP), polyvinyl acetate phthalate (PVAP), and combinations thereof.

The polymer coating comprises about 1% to about 30%, by weight, of the uncoated naltrexone-ion exchange resin complex granule e.g., about 3% to about 25% by weight, about 10% to about 20% by weight, or about 15% by weight of the uncoated drug resin complex.

### Liquid Resin Formulations

The coated naltrexone-ion exchange resins particles are formulated into finished ingestible dosage forms such as a liquid suspension. It has also been observed that for use in liquid compositions, the film forming coating for the drug-ion exchange resin complex, when formulated into a liquid suspension, does not produce undesirable agglomerations and color migration of the suspended particles in the liquid in the presence of a colorant which is used in medicines to be taken by children.

Suitable flavorings include both natural and artificial flavors, and mints such as peppermint, menthol, artificial vanilla, cinnamon, various fruit flavors, both individual and mixed, essential oils (i.e. thymol, eucalyptol, menthol and methyl salicylate) and the like are contemplated. The amount of flavoring employed is normally a matter of preference subject to such factors as flavor type, individual flavor, and strength desired. Thus, the amount may be varied in order to obtain the result desired in the final product. The flavorings are generally utilized in amounts that will vary depending upon the individual flavor, and may, for example, range in amounts of about 0.01 to about 3% by weight per volume of the final composition weight.

The colorants include the pigments such as titanium dioxide that may be incorporated in amounts of up to about 1% by weight per volume, and preferably up to about 0.6% by weight per volume. Also, the colorants may include dyes suitable for food, drug and cosmetic applications, and known as D&C and F.D. & C. dyes and the like. The materials acceptable for the foregoing spectrum of use are preferably water-soluble. Illustrative examples include indigoid dye, known as F.D. & C. Blue No. 2, which is the disodium salt of 5,5′indigotindisulfonic acid. Similarly, the dye known as F.D. & C. Green No. 1 comprises a triphenylmethane dye and is the monosodium salt of 4-[4-N-ethyl p-sulfobenzylamino)diphenylmethylene]-[1-(N-ethyl-N-p-sulfoniumbenzyl)-2,5-cyclohexadienimine]. A full recitation of all F.D. & C. and D. & C. and their corresponding chemical structures may be found in the Kirk-Othmer Encyclopedia of Chemical Technology, in Volume 5, at Pages 857-884, which text is accordingly incorporated herein by reference.

Suitable oils and fats that are usable would include partially hydrogenated vegetable or animal fats, such as coconut oil, palm kernel oil, beef tallow, and lard. These ingredients are generally utilized in amounts with respect to the comestible product of up to about 7.0% by weight, and preferably up to about 3.5% by weight of the final product.

Wetting agents also may be employed to facilitate the dispersion of any hydrophobic ingredients. The concentration of wetting agents in the composition should be selected to achieve optimum dispersion of the ingredient within the composition with the lowest feasible concentration of wetting agent. It should be appreciated that an excess concentration of wetting agent may cause the composition, as a suspension, to flocculate. Suitable wetting agents are listed in the US Pharmacopeia 29.

### Naltrexone HCl

Naltrexone HCl can be produced from noroxymorphone by various direct and indirect alkylation methods. One method is by direct alkylation of noroxymorphone with cyclopropylmethylbromide. This process has been disclosed in WO 91/05768, the disclosure of which is incorporated by reference herein. WO 2008/034973, the disclosure of which is incorporated by reference herein, describes a process for obtaining naltrexone in 88.6% yield by reacting noroxymorphone hydrochloride with cyclopropylmethylbromide in dimethylacetamide in the presence of sodium hydrogen carbonate. WO 2008/138605, the disclosure of which is incorporated by reference herein, describes N-alkylation of noroxymorphone with cyclopropylmethylbromide in N-methyl-pyrrolidone in the presence of sodium hydrogen carbonate. WO 2010/039209, the disclosure of which is incorporated by reference herein, describes N-alkylation of noroxymorphone with cyclopropylmethylbromide in the presence of a protic solvent. Specific examples in WO 2010/039209 describe the addition of water, isopropanol or ethanol as the protic solvent.

Naltrexone may be synthesized by producing naltrexone[17-(cyclopropylmethyl)-4,5α-epoxy-3,14-dihydroxy-morphinan-6-one] from noroxymorphone[4,5-α-epoxy-3,14-dihydroxy-morphinan-6-one] by alkylation with a cyclopropylmethyl halide. Naltrexone can be prepared by various processes, for example those descried in WO 91/05768, WO 2008/034973, WO 2008/138605, WO 2010/039209, and WO 2010/039209, the disclosures of each of which are incorporated by reference herein.

### Example 1

This example provides a liquid resin naltrexone formulation to achieves a liquid resin extended-release oral naltrexone formulation suspension comprising from about 1.0 mg/ml to about 10.0 mg/ml in a resin, wherein no more than 25% of the total dose administered is released within one hour and wherein no more than 60% of the total dose administered is released within two hours. Specifically, a naltrexone resin complex was created according to the formulation shown in Table 1.

TABLE 1
Naltrexone resin complex

| Ingredient | Quantity | |
|---|---|---|
| Naltrexone HCl | 0.5 | kg |
| Amberlite IRP69 (polystyrene sulfonate) | 1.0 | kg |
| Purified water | 10.0 | kg |

Purified water was dispensed into a container and naltrexone HCl was added and mixed until dissolved. Sodium polystyrene sulfonate (Amberlite IRP69 resin) was added to the tank and mixed. The resulting dispersion was passed through filter paper and the retained drug resin complex was washed with purified water. The drug resin complex was loaded into an oven for drying until the moisture was below 10%.

The formulation in Table 1 was used to create Naltrexone resin complex granules summarized below in Table 2. Polyethylene glycol was added into purified water and mixed until dissolved. The resulting PEG solution was sprayed on to the wet resin complex and granulated using a high shear granulator. The wet granules were dried in an oven until the moisture reached between 15% to 20%. The partially dried granules were milled using a co-mill fitted with 400 micron mesh screen. The pass-through granules were dried until the moisture reached below 7%.

TABLE 2
Composition of Naltrexone resin complex granules

| Ingredient | Quantity |
|---|---|
| Naltrexone resin complex | 675 g |
| Polyethylene glycol | 122 g |
| Purified water | 304 |

The resulting complex was coated to form the formulation summarized below in Table 3. Acetone and purified water were added to a container and mixed well. Polyethylene glycol was added to the container and mixed until dissolved. Cellulose acetate butyrate was added and mixed until dissolved. The coating solution was sprayed onto the naltrexone resin complex granules of Table 2 using a fluid bed apparatus to achieve a weight gain of 15%.

TABLE 3
Composition of Coated naltrexone resin complex

| Ingredient | Quantity | |
|---|---|---|
| Naltrexone resin complex granules | 600 | g |
| Cellulose acetate butyrate | 78.9 | g |
| Polyethylene glycol | 15.8 | g |
| Acetone | 1620 | g |

| | |
|---|---|
| Purified water | 180 |

The resulting formulation was then used to create a Naltrexone Extended-Release Suspension, summarized in Table 4 below. The placebo base was prepared by adding sucrose to the main container containing purified water and mixed until dissolved followed by adding citric acid. Starch was dispersed in the container and mixed well. In a separate container, methylparaben and propylparaben were added into propylene glycol and mixed until dissolved completely. Xanthan gum was added to the preservative solution and mixed well until it was uniformly dispersed. This gum dispersion was slowly added to the main container and mixed well. Sodium metabisulfite and Tween 80 were added into the main container and mixed until dissolved. Purified water was added to the main container to make up the final volume to 1.2 L. To the main container, coated naltrexone resin complex was added under continuous mixing to form the Naltrexone ER Suspension.

TABLE 4
Composition of Naltrexone Extended-Release Suspension

| | |
|---|---|
| Ingredient | Quantity |

Placebo base

| | | |
|---|---|---|
| Purified water | 650 | g |
| Sucrose | 240 | g |
| Citric acid anhydrous | 0.24 | g |
| Starch | 25.44 | g |
| Propylene glycol | 120 | g |
| Methylparaben | 2.16 | g |
| Propylparaben | 0.24 | g |
| Xanthan gum | 1.8 | g |
| Sodium metabisulfite | 1.2 | g |
| Tween 80 | 1.2 | g |

| | |
|---|---|
| Purified water | QS to 1.2 L |

Naltrexone Extended-Release suspension

| | | |
|---|---|---|
| Placebo base | 900 | mL |
| Coated naltrexone resin | 22.1 | g |

### Example 2—In Vitro Drug Release

A coated Naltrexone resin complex was prepared as per example 1. In this example, cherry flavor and FD&C red 40 were used to prepare the final suspension. Ascorbic acid was used as antioxidant instead of sodium metabisulfite.

TABLE 5
Naltrexone Extended-Release Suspension

| | |
|---|---|
| Ingredient | Quantity |

Suspension base

| | | |
|---|---|---|
| Purified water | 800 | g |
| Sucrose | 320 | g |
| Citric acid anhydrous | 0.16 | g |
| Starch | 33.92 | g |
| Propylene glycol | 160 | g |
| Methylparaben | 2.88 | g |
| Propylparaben | 0.32 | g |
| Xanthan gum | 2.4 | g |
| Ascorbic acid | 0.96 | g |
| Tween 80 | 1.6 | g |

| | |
|---|---|
| Purified water | QS 1600 mL |

Naltrexone ER Suspension

| | | |
|---|---|---|
| Suspension base | 500 | ml |
| Coated naltrexone | 12.29 | g |
| resin (from example 1) | | |
| FD&C red 40 | 0.05 | g |
| Cherry flavor | 1.0 | g |

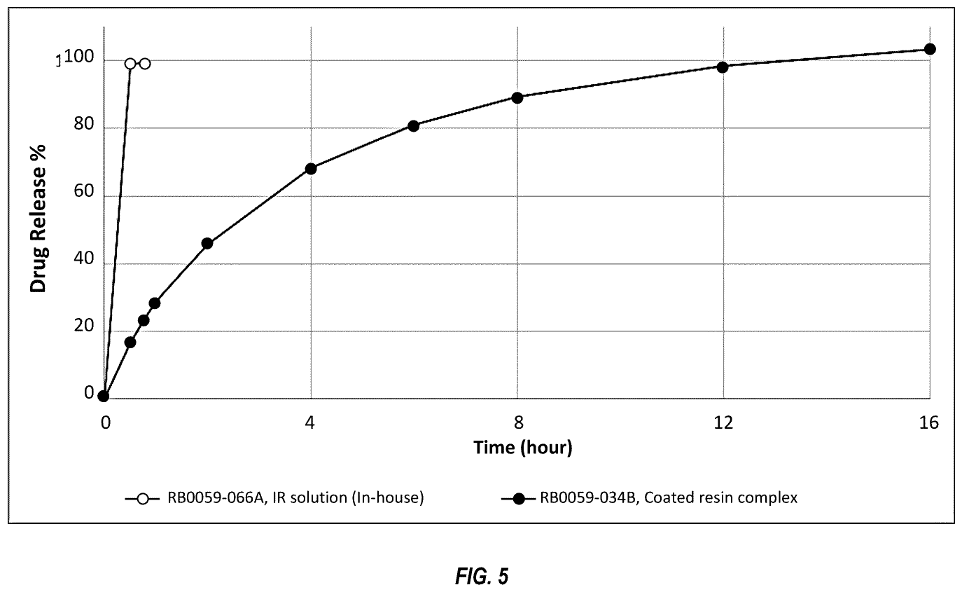

Purified water was added to a main container followed by sucrose and mixed until dissolved. Citric acid was added to the main container and mixed until dissolved. Starch was then dispersed in the container and mixed well. In a separate container, methylparaben and propylparaben were added into propylene glycol and mixed until dissolved completely. Xanthan gum was added to the preservative solution and mixed well until it was uniformly dispersed. The xanthan gum dispersion was slowly added to the main container and mixed well. Ascorbic acid and Tween 80 were added into the main container and mixed until dissolved. Purified water was added to the main container to make up the final suspension base volume to 1.6 L. To 500 mL of the suspension base, FD&C red number 40, Cherry flavor and coated naltrexone resin was added under continuous mixing to form the Naltrexone ER Suspension. The Naltrexone ER Suspension was tested for in vitro drug release using USP apparatus II, 0.4M KH₂PO₄, 900 mL, 50 rpm, 37±0.5° C. The results are summarized in FIG. 5.

### Example 3—Coated Drug Ion Exchange Particles

Naltrexone resin complex granules were prepared as per example 1. In this example cellulose acetate butyrate and hypromellose phthalate were used to coat the naltrexone resin complex granules. The formulation was prepared according to Table 6.

TABLE 6
Composition of Coated naltrexone resin complex

| Ingredient | Quantity | |
|---|---|---|
| Naltrexone resin complex granules | 500 | g |
| Cellulose acetate butyrate | 39 | g |
| Hypromellose phthalate | 39 | g |
| Acetone | 1407.9 | g |
| Purified water | 74.1 | g |

To a container, acetone and purified water were added and mixed well. Polyethylene glycol was added to the container and mixed until dissolved. Cellulose acetate butyrate was added and mixed until dissolved. Hypromellose phthalate was then added and mixed until dissolved. The coating solution was sprayed on to naltrexone resin complex granules using fluid bed apparatus to achieve a weight gain of 15% w/w.

### Example 4

Naltrexone resin complex granules were prepared as per example 1. In this example ethyl cellulose (EC) was used to coat the naltrexone resin complex granules. The formulation was prepared according to Table 7.

TABLE 7
Composition of Coated naltrexone resin complex

| Ingredient | Quantity | |
|---|---|---|
| Naltrexone resin complex granules | 500 | g |
| Ethyl cellulose | 65.7 | g |
| Myvacet 9-45 | 13.2 | g |
| Acetone | 1349.5 | g |
| Purified water | 150 | g |

To a container, acetone and purified water were added and mixed well. Myvacet 9-45 was added mixed until dissolved. Ethyl cellulose was added and mixed until dissolved. A total 1502 g of coating solution was sprayed onto the naltrexone resin complex granules using fluid bed apparatus to achieve a weight gain of 15% w/w.

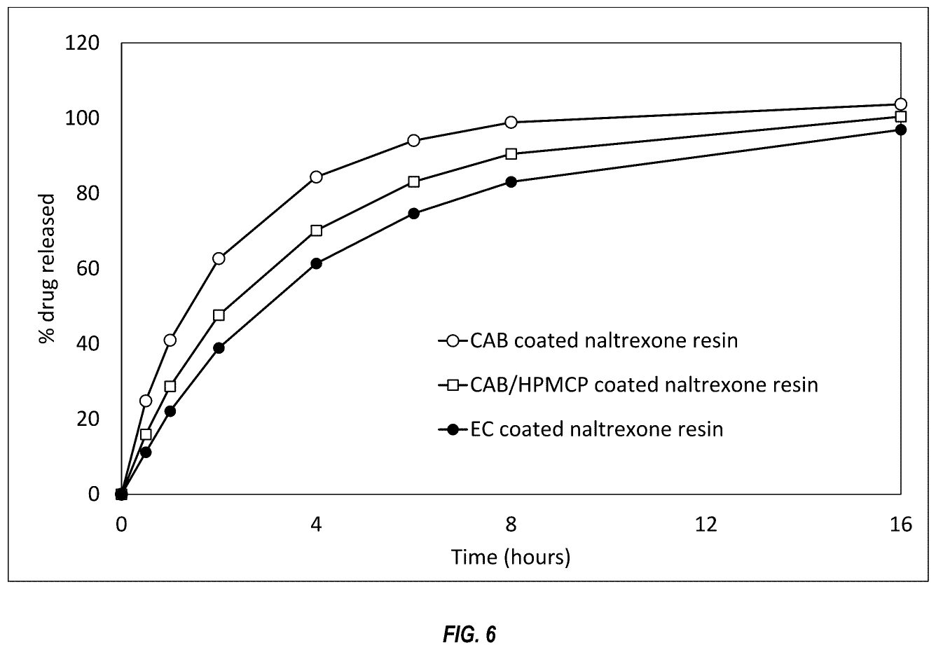

The coated naltrexone resin prepared as per example 4 was tested for in-vitro drug release using USP apparatus II, 0.4M KH₂PO₄, 900 mL, 50 rpm, 37±0.5° C. The results are summarized in FIG. 6.

### Example 5

A Naltrexone resin complex was prepared according to the formulation shown in Table 8.

TABLE 8
Naltrexone resin complex

| Ingredient | Quantity | |
|---|---|---|
| Naltrexone HCl | 0.5 | kg |
| Amberlite IRP69 | 1.0 | kg |
| Purified water | 10 | kg |

Purified water was dispensed into a container and naltrexone HCl was added and mixed until dissolved. Sodium polystyrene sulfonate (Amberlite IRP69 resin) was added to the tank and mixed. The supernatant from the resulting dispersion was decanted followed by washing with purified water and finally the dispersion was filtered using a filter housing. The wet drug resin complex was dried using a fluid bed processor until the moisture content was about 15%.

The formulation in Table 8 was used to prepare Naltrexone resin complex granules summarized below in Table 9. EDTA was added into purified water and mixed until dissolved. Polyethylene glycol was added to the container and mixed until dissolved. The resulting PEG solution was sprayed on to the wet resin complex and granulated using a high shear granulator. The wet granules were dried using fluid bed processor until the moisture reaches between 15% to 20%. The partially dried granules were milled using a co-mill fitted with about 813-micron mesh screen. The pass-through granules were dried using fluid bed processor until the moisture reached below 7%.

TABLE 9
Composition of Naltrexone resin complex granules

| Ingredient | Quantity | |
|---|---|---|
| Naltrexone resin complex | 1300 | g |
| EDTA | 4 | g |
| Polyethylene glycol | 244 | g |
| Purified water | 366 | g |

The resulting complex was coated to form the formulation summarized below in Table 10. Acetone and purified water were added to a container and mixed well. Polyethylene glycol was added to the container and mixed until dissolved. Cellulose acetate butyrate was added and mixed until dissolved. The coating solution was sprayed onto the naltrexone resin complex granules of Table 9 using a fluid bed processor to achieve a weight gain of 15% w/w.

TABLE 10
Composition of Coated naltrexone resin

| Ingredient | Quantity | |
|---|---|---|
| Naltrexone resin complex granules | 500 | g |
| Cellulose acetate butyrate | 71 | g |
| Polyethylene glycol | 7.1 | g |
| Acetone | 1424 | g |
| Purified water | 75 | g |

The resulting coated naltrexone resin was used to prepare a Naltrexone Extended-Release Suspension, summarized in Table 11 below. The suspension was prepared by adding glycerin to a container and heated to 45-60° C. Methylparaben and propylparaben were added to the container and mixed until dissolved. Xanthan gum was added to the preservative solution and mixed until it was uniformly dispersed (gum dispersion). To the main container containing purified water, sucrose, EDTA and citric acid were added and mixed until dissolved. Starch was added to the main container and mixed until uniformly dispersed. The gum dispersion was slowly added to the main container and mixed well. Tween 80, color and flavor were added to the main container and mixed well. The coated naltrexone resin was added to the main container and purified water was added to make up the final suspension volume to 1.6 L.

TABLE 11
Composition of Naltrexone Extended-Release Suspension

| Ingredient | Quantity | |
|---|---|---|
| Purified water | 676 | g |
| Sucrose | 320 | g |
| Citric acid anhydrous | 0.32 | g |
| EDTA | 1.6 | g |
| Starch | 33.9 | g |
| Glycerin | 240 | g |
| Methylparaben | 2.88 | g |
| Propylparaben | 0.32 | g |
| Xanthan gum | 2.4 | g |
| Tween 80 | 1.2 | g |
| Coated naltrexone resin | 38.46 | g |
| FD & C red 40 | 0.08 | g |
| Cherry flavor | 1.6 | g |

| | |
|---|---|
| Purified water | QS to 1.6 L |

### Example 6

Coated naltrexone resin was prepared as per example 5. The naltrexone extended-release suspension was prepared as summarized in Table 12 below. The suspension was prepared by adding glycerin to a container and heated to 45-55° C. Methylparaben and propylparaben were added to the container and mixed until dissolved. Xanthan gum was added to the preservative solution and mixed until it was uniformly dispersed (gum dispersion). To the main container containing purified water, EDTA and citric acid were added and mixed until dissolved. Tween 80 was added and mixed well. Starch and sucrose were added together to the main container and mixed until uniformly dispersed. The gum dispersion was slowly added to the main container under continuous mixing. Color and flavor were added to the main and mixed well. The coated naltrexone resin was added to the main container and purified water was added to make up the final suspension volume to 1.6 L.

TABLE 12
Composition of Naltrexone Extended-Release Suspension

| Ingredient | Quantity | |
|---|---|---|
| Purified water | 680 | g |
| Sucrose | 320 | g |
| Citric acid anhydrous | 0.32 | g |
| EDTA | 1.6 | g |
| Starch | 33.9 | g |
| Glycerin | 960 | g |
| Methylparaben | 2.88 | g |
| Propylparaben | 0.32 | g |
| Xanthan gum | 2.4 | g |
| Tween 80 | 1.2 | g |
| Coated naltrexone resin | 38.46 | g |
| FD & C red 40 | 0.08 | g |
| Cherry flavor | 1.6 | g |

| | |
|---|---|
| Purified water | QS to 1.6 L |

### Example 7

This example provides a drug release profile, over time, of an immediate release naltrexone (IR, steep blue line) which is an uncoated resin suspension and naltrexone in solution, and the extended-release formulation described in Example 1 called a “coated resin complex” and the red line. FIG. 5 shows the comparison of release kinetics between the two formulations.

### Example 8

A Naltrexone resin complex was prepared according to the formulation shown in

| Ingredient | Quantity (kg) |
|---|---|
| Naltrexone HC1 | 1.5 |
| Amberlite IRP69 | 3.0 |
| Purified water | 30.0 |

Purified water was dispensed into a container and naltrexone HCl was added and mixed until dissolved. Sodium polystyrene sulfonate (Amberlite IRP69 resin) was added to the tank and mixed. The supernatant from the resulting dispersion was decanted followed by washing with purified water and finally the dispersion was filtered using a filter housing. The wet drug resin complex was dried using a fluid bed processor until the moisture content was about 15%.

The formulation in Table 13 was used to prepare Naltrexone resin complex granules summarized below in Table 14. EDTA was added into purified water and mixed until dissolved. Polyethylene glycol (PEG) was added to the container and mixed until dissolved. PEG solution was sprayed on to the wet resin complex and granulated using a high shear granulator. The wet granules were dried using fluid bed processor until the moisture reached between 15% to 25%. The partially dried granules were passed through #20 mesh screen and dried using the fluid bed dryer until the moisture reached below 7%. These dried granules were milled using a co-mill fitted with 813-micron mesh screen.

TABLE 14
Composition of Naltrexone resin complex granules

| Ingredient | Quantity (g) |
|---|---|
| Naltrexone resin complex | 4300 |
| EDTA | 16 |
| Polyethylene glycol | 946 |
| Purified water | 1.42 |

The resulting complex was coated to different weight gains using the coating composition shown in Table 15. Acetone and purified water were added to a container and mixed well. Polyethylene glycol was added to the container and mixed until dissolved. Cellulose acetate butyrate was added and mixed until dissolved. The coating solution was sprayed onto the naltrexone resin complex granules of Table 14 using a fluid bed processor to achieve a coating weight gain of 15% w/w and 20% w/w.

TABLE 15
Composition of Coating Solution

| Ingredient | Quantity (g) |
|---|---|
| Cellulose acetate butyrate | 96 |
| Polyethylene glycol | 9.6 |
| Acetone | 1906.1 |
| Purified water | 100.3 |

The resulting coated naltrexone resin was used to prepare a Naltrexone Extended-Release Suspension, summarized in Table 16 below. To the main container containing purified water, Tween 80, propyl gallate, sodium benzoate, EDTA and citric acid anhydrous were added and mixed until dissolved. Xanthan gum, starch and sucrose were mixed together and added into the main tank and mixed until uniformly dispersed. Color solution was added into the main tank while mixing. The coated naltrexone resin and flavor was added to the main container and purified water was added to make up the final suspension volume to 4 L.

TABLE 16
Composition of Naltrexone Extended-Release Suspension

| | |
|---|---|
| Formulation 1 | Formulation 2 |

| | |
|---|---|
| Ingredient | Quantity (g) |

| | | |
|---|---|---|
| Purified water | 2680 | 2680 |
| Sucrose | 800 | 800 |
| Citric acid anhydrous | 16 | 16 |
| EDTA | 3.8 | 3.8 |
| Starch | 84.39 | 84.39 |
| Sodium benzoate | 8 | 8 |
| Xanthan gum | 6 | 6 |
| Tween 80 | 4 | 4 |
| Propyl gallate | 0.8 | 0.8 |
| Coated naltrexone resin | 85.1 | 89.3 |
| FD & C red 40 | 0.2 | 0.2 |
| Cherry flavor | 4 | 4 |
| Purified water | QS to 4 L | QS to 4 L |

A clinical pharmacokinetic study was conducted comparing Formulation 1 and Formulation 2 in Table 16. The study was an open-label, single-dose, randomized, three-period, three-treatment, three-sequence, crossover, comparative bioavailability study in healthy adults (n=18) under fasted condition.

Treatment A—Formulation 1: 10 mL of 5 mg/mL oral suspension (Total dose=50 mg naltrexone HCl) administered after an overnight fast of at least 10 hours.

Treatment B—Formulation 2: 10 mL of 5 mg/mL oral suspension (Total dose=50 mg naltrexone HCl) administered after an overnight fast of at least 10 hours.

Treatment C—reference product: Naltrexone HCl Tablets, 50 mg (Mallinckrodt™ SpecGx LLC): one (1) 50 mg tablet administered after an overnight fast of at least 10 hours.

Both naltrexone and its active metabolite (6-β-Naltrexol) were analyzed.

The AUCₜ, AUC_{∞}, Cₘₐₓ, Tₘₐₓ and T_{1/2} values obtained for Naltrexone and 6-β-Naltrexol are shown in Table 17:

TABLE 17
Pharmacokinetic Parameters

| | | | | | |
|---|---|---|---|---|---|
| | AUCₜ | AUC_{∞} | Cₘₐₓ | Tₘₐₓ | T_{1/2} |
| Product | (hr · ng/mL) | (hr · ng/mL) | (ng/mL) | (hrs) | (hrs) |

Naltrexone

| | | | | | |
|---|---|---|---|---|---|
| Formulation 1 (A) | 29.705 | 31.762 | 2.904 | 5.91 | 7.07 |
| Formulation 2 (B) | 28.211 | 32.450 | 2.088 | 5.75 | 11.10 |
| Reference (C) | 20.859 | 21.989 | 8.199 | 0.78 | 5.44 |

6-β-Naltrexol

| | | | | | |
|---|---|---|---|---|---|
| Formulation 1 (A) | 790.770 | 905.573 | 34.871 | 6.21 | 14.84 |
| Formulation 2 (B) | 690.615 | 861.430 | 24.078 | 8.97 | 16.42 |
| Reference (C) | 779.762 | 905.573 | 102.753 | 0.91 | 14.01 |

The Mean Plasma Naltrexone and Mean Plasma 6-β-Naltrexol Concentrations are shown in Tables 18 and 19, respectively:

TABLE 18
Mean Plasma Naltrexone Concentrations

| Time | Formulation 1 | Formulation 2 | Reference |
|---|---|---|---|
| (hrs) | (ng/mL) | (ng/mL) | (ng/mL) |
| 0 | 0.000 | 0.000 | 0.000 |
| 0.25 | 0.247 | 0.217 | 1.313 |
| 0.5 | 0.741 | 0.679 | 5.855 |
| 0.75 | 0.849 | 0.722 | 6.712 |
| 1 | 0.834 | 0.733 | 6.257 |
| 1.5 | 0.977 | 0.808 | 4.734 |
| 2 | 1.160 | 0.876 | 3.758 |
| 2.5 | 1.336 | 0.957 | 3.129 |
| 3 | 1.429 | 0.968 | 2.475 |
| 3.5 | 1.467 | 0.997 | 2.036 |
| 4 | 1.495 | 1.020 | 1.679 |
| 6 | 2.833 | 2.019 | 0.843 |
| 8 | 1.952 | 1.570 | 0.455 |
| 10 | 1.552 | 1.340 | 0.391 |
| 12 | 1.319 | 1.177 | 0.325 |
| 16 | 0.771 | 0.845 | 0.177 |
| 24 | 0.312 | 0.486 | 0.038 |
| 36 | 0.088 | 0.180 | 0.000 |
| 48 | 0.021 | 0.047 | 0.000 |

TABLE 19
Mean Plasma 6-β-Naltrexol Concentrations

| Time | Formulation 1 | Formulation 2 | Reference |
|---|---|---|---|
| (hrs) | (ng/mL) | (ng/mL) | (ng/mL) |
| 0 | 0.000 | 0.000 | 0.000 |
| 0.25 | 2.454 | 2.121 | 12.150 |
| 0.5 | 7.811 | 6.604 | 68.629 |
| 0.75 | 10.381 | 8.384 | 91.124 |
| 1 | 11.135 | 8.885 | 90.165 |
| 1.5 | 13.860 | 10.644 | 74.653 |
| 2 | 17.764 | 12.105 | 66.465 |
| 2.5 | 20.812 | 13.924 | 59.188 |
| 3 | 22.888 | 14.713 | 53.147 |
| 3.5 | 24.649 | 16.239 | 47.947 |
| 4 | 26.512 | 17.596 | 42.929 |
| 6 | 33.771 | 22.778 | 33.900 |
| 8 | 30.129 | 21.856 | 25.441 |
| 10 | 27.771 | 21.511 | 21.535 |
| 12 | 27.088 | 21.312 | 19.124 |
| 16 | 23.094 | 20.046 | 15.640 |
| 24 | 17.384 | 17.689 | 10.878 |
| 36 | 8.346 | 9.344 | 5.272 |
| 48 | 5.182 | 5.678 | 3.458 |

The Pharmacokinetic Profile for Naltrexone Formulations 1 and 2, and the commercial product (Naltrexone HCl Tablets, 50 mg; Mallinckrodt™, SpecGx LLC) is shown in FIG. 8. The results show overall that the Formulations of the present disclosure displayed better bioavailability (˜50% more bioavailable) compared to the commercial immediate release tablet. For naltrexone there was an ˜65% reduction in Cₘₐₓ for Formulation 1 and ˜75% reduction in Cₘₐₓ for Formulation 2. This significantly reduces side effects caused by abrupt rise of plasma concentration from immediate release product.

The Tₘₐₓ was delayed from less than 1 hour for reference intermediate release (IR) tablet to ˜6 hours for both Formulations 1 & 2, showing the extended-release nature of the test products. The duration of effectiveness for the Formulations 1 & 2 was prolonged compared to immediate release reference product due to slower rise and drop of plasma concentration. Formulations 1 & 2 maintain plasma concentration above 0.75 ng/mL for ˜16 hours after oral ingestion, however, it is only ˜6 hours for the reference product.

For 6-β-Naltrexol there was a ˜66% reduction in Cₘₐₓ for Formulation 1 and ˜77% reduction in Cₘₐₓ for Formulation 2. This significantly reduces side effects caused by abrupt rise of plasma concentration from immediate release product. Similar to the pattern for naltrexone's plasma profile, the active metabolite, 6-β-Naltrexol, derived from Formulations 1 & 2, exhibited lower Cₘₐₓ and longer Tₘₐₓ compared to reference product. However, comparable bioavailability was observed for Formulations 1 and 2, and the reference product.

The biological half-life for 6-O-Naltrexol is comparable among Formulations 1 & 2 and the reference product. The plasma profile of the extended-release Formulations 1 & 2 exhibit higher concentration than the reference product from 10 hours onward after oral ingestion.

### Example 9 (Prophetic)

This example provides a clinical protocol in which the liquid resin extended-release oral naltrexone formulation will be studied in children having autistic spectrum disorders (ASD). The drug product is an extended-release oral suspension formulation of naltrexone HCl. The active ingredient, naltrexone HCl is complexed to sodium polystyrene sulfonate (DuPont™ Amberlite™ IRP69), a cation exchange resin to form the naltrexone resin complex. The drug resin complex is further granulated followed by coating with an extended-release polymer (polyethylene glycol). The final suspension is prepared by dispersing the coated naltrexone resin particles into the suspension base to provide the dose strength of 22.6 mg/5 mL or 11.3 mg/5 mL (equivalent to 5 mg/mL or 2.5 mg/mL naltrexone HCl). The final suspension is packaged into 8-ounce (oz) (240 mL) high density polyethylene (HDPE) bottles with 24-mm white Child Resistant Closures (CRC). The composition of the placebo is qualitatively the same as the suspension of 25 mg/5 mL strength but without naltrexone HCl active pharmaceutical ingredient.

### Primary Outcome Measures:

1. The number of Participants with Reduction in ASD Symptom Severity as Defined by the Social Responsiveness Scale (SRS);
2. The number of participants with reduction in ASD symptom severity defined as a reduction in Social Responsiveness Scale (SRS) score from baseline of greater than or equal to 30%.

The SRS is a 65-item rating scale completed by an informant to measure the severity of autism spectrum symptoms as they occur in natural settings.

### Secondary Outcome Measures:

1. The number of Participants with Reduction in ASD Symptom Severity as Defined by the NIMH Clinical Global Impression for Pervasive Developmental Disorders (CGI-PDD) Improvement Score;
2. The number of participants with reduction in ASD symptom severity defined as an NIMH Clinical Global Impression (CGI) Pervasive Developmental Disorder (PDD) Improvement score less than or equal to 2. The CGI-Improvement is a clinician-rated measure of improvement. Scores range from 1 (very much improved) to 7 (very much worse) for PDD.

### Inclusions

Male and female outpatients >12 years of age.
Diagnostic confirmation of ASD as confirmed by gold standard clinical interview using Diagnostic and Statistical Manual of Mental Disorders 5th Edition (DSM-5) criteria and administration of the Autism Diagnostic Observation Schedule-2, Module 3 or 4.
Subjects and/or their legal representative must have a level of understanding sufficient to communicate intelligently with the investigator and study coordinator, and to cooperate with all tests and examinations required by the protocol.
Subjects and/or their legal representative must be considered reliable reporters.
Each subject and/or their authorized legal representative must understand the nature of the study. The subject and/or their legal representative must sign an informed consent document.
Subject must be able to participate in mandatory blood draws.
Subject must be able to drink measured liquid formulation.
Subjects with mood, anxiety, or disruptive behavior disorders will be allowed to participate in the study provided they do not meet any exclusionary criteria.

### Exclusions

IQ<85.
Allergy or hypersensitivity to naltrexone
Total lack of spoken language.
Relevant gastroesophageal reflux disease as per investigator
Clinically unstable psychiatric conditions or judged to be at serious suicidal risk.
Current diagnosis of a psychotic disorder or unstable bipolar disorder.
History of recent or current (past 30 days) clinically significant depressive or anxiety disorder that warrants treatment.
Current diagnosis of schizophrenia.
History of substance use (except nicotine or caffeine) within past 3 months
Serious, stable or unstable systemic illness including hepatic, renal, gastroenterological, respiratory, cardiovascular (including ischemic heart disease), endocrinologic, neurologic, immunologic, or hematologic disease.
Subjects with severe hepatic impairment (LFTs>3 times ULN) and those with severely impaired renal function (eGFR<30).
Uncorrected hypothyroidism or hyperthyroidism.
Subjects with untreated and/or unstable diabetes.
Non-febrile seizures without a clear and resolved etiology.
Pregnant or nursing females.
Severe allergies or multiple adverse drug reactions.
A non-responder or history of intolerance to memantine, after treatment at adequate doses as determined by the clinician.
Investigator and his/her immediate family defined as the investigator's spouse, parent, child, grandparent, or grandchild.

### Primary Outcome Measures:

### 1. Change in Maladaptive Behaviors

Demonstrate a change in frequency and intensity of maladaptive behaviors as measured by the Aberrant Behavior Checklist (ABC) This checklist consists of 20 questions relating to behavior and the reported total score is on a scale from 0 to 60. A lower score can be interpreted as less frequent and/or less intense presentation of the undesirable behavior. The below values are the difference in ABC scores from baseline to 8 weeks. A negative difference indicates improved behavior.

### 2. Primary Safety Endpoints

Number of serious adverse events

### Secondary Outcome Measures:

### 1. Change in Aggressive Behavior

Demonstrate a trend towards reduced aggressive behavior as measured by Overt Aggression Scale (OAS). It consists of a scale from 0-40, and a lower score can be interpreted as less frequent and/or less intense presentation of the undesirable behavior. Reported is the mean difference in scores from baseline to 8 weeks. A positive score indicates more aggressive behavior and a negative score indicates less aggressive behavior.

We claim:

1. A liquid resin extended-release oral naltrexone formulation suspension comprising from about 1.0 mg/ml to about 10.0 mg/ml naltrexone in a resin, wherein no more than 30% of the total naltrexone dose administered is released within one hour and wherein no more than 60% of the total naltrexone dose administered is released within two hours.

2. The liquid resin extended-release oral naltrexone formulation suspension of claim 1 comprises (a) a resinous core comprising naltrexone HCl and an ion exchange resin selected from the group consisting of polystyrene, a co-polymer of polystyrene and divinylbenzene; (b) a material coating the resinous core to form a particle, wherein the coating material is selected from the group consisting of cellulose acetate, cellulose diacetate, cellulose triacetate, cellulose acetate butyrate, cellulose acetate phthalate (CAP), cellulose acetate propionate, cellulose acetate phthalate (CAP), hypromellose phthalate (hydroxypropylmethylcellulose phthalate; HPMCP), polyvinyl acetate phthalate (PVAP); cellulose acetate trimellitate; hydroxypropylmethylcellulose acetate succinate; sodium alginate; alginic acid, shellac and combinations thereof, and (c) an aqueous thickened flavored and sweetened liquid for suspending the particles.

3. The liquid resin extended-release oral naltrexone liquid suspension formulation of claim 1, wherein the ion exchange resin is polystyrene.

4. The liquid resin extended-release oral naltrexone liquid suspension formulation of claim 1, wherein the material coating the resinous core to form a particle is a cellulose acetate selected from the group consisting of cellulose acetate, cellulose diacetate, cellulose triacetate, cellulose acetate butyrate, cellulose acetate phthalate (CAP), cellulose acetate propionate, cellulose acetate phthalate (CAP).

5. The liquid resin extended-release oral naltrexone liquid suspension formulation of claim 1, wherein the aqueous thickened flavored and sweetened liquid for suspending the particles comprises water, from about 300 to about 500 g/L of sucrose, from about 0.1 to about 0.3 g/L citric acid (anhydrous), from about 30 to about 55 g/L starch, from about 150 to about 350 g/L propylene glycol, from about 3 to about 5 g/L parabens, from about 2.0 to about 5.0 xanthan gum, from about 0.5% to about 2% Tween 80.

6. A method for treating a child with an autism-type disorder with approximately a teaspoon (about 5 ml) of a liquid resin extended-release oral naltrexone formulation suspension comprising from about 1.0 mg/ml to about 10.0 mg/ml naltrexone in a resin, wherein no more than 30% of the total naltrexone dose administered is released within one hour and wherein no more than 60% of the total naltrexone dose administered is released within two hours.

7. The method of claim 6 wherein the liquid resin formulation comprises (a) a resinous core comprising naltrexone HCl and an ion exchange resin selected from the group consisting of polystyrene, a co-polymer of polystyrene and divinylbenzene; (b) a material coating the resinous core to form a particle, wherein the coating material is selected from the group consisting of cellulose acetate, cellulose diacetate, cellulose triacetate, cellulose acetate butyrate, cellulose acetate phthalate (CAP), cellulose acetate propionate, cellulose acetate phthalate (CAP), hypromellose phthalate (hydroxypropylmethylcellulose phthalate; HPMCP), polyvinyl acetate phthalate (PVAP); cellulose acetate trimellitate; polymethacrylates (e.g., Eudragit® L series, Eudragit® S series, Eudragit® FS, Kollicoat® MAE 100P, Acryl-EZE® 93A, Acryl-EZE® MP); hydroxypropylmethylcellulose acetate succinate (HPMC AS; Aqoat®); sodium alginate; alginic acid, shellac and combinations thereof, and (c) an aqueous thickened flavored and sweetened liquid for suspending the particles.

8. The method of claim 6 wherein the liquid resin extended-release oral naltrexone formulation suspension is administered qAM (upon waking).

9. A method for treating a child with a rare pediatric disease Schaff-Yang Syndrome (SYS) or Bosch-Boonstra-Schaaf Optic Atrophy Syndrome (BBSOAS) with approximately a teaspoon (about 5 ml) of a liquid resin extended-release oral naltrexone formulation suspension comprising from about 1.0 mg/ml to about 10.0 mg/ml naltrexone in a resin, wherein no more than 30% of the total naltrexone dose administered is released within one hour and wherein no more than 60% of the total naltrexone dose administered is released within two hours.

10. The method of claim 9 wherein the liquid resin formulation comprises (a) a resinous core comprising naltrexone HCl and an ion exchange resin selected from the group consisting of polystyrene, a co-polymer of polystyrene and divinylbenzene; (b) a material coating the resinous core to form a particle, wherein the coating material is selected from the group consisting of cellulose acetate, cellulose diacetate, cellulose triacetate, cellulose acetate butyrate, cellulose acetate phthalate (CAP), cellulose acetate propionate, cellulose acetate phthalate (CAP), hypromellose phthalate (hydroxypropylmethylcellulose phthalate; HPMCP), polyvinyl acetate phthalate (PVAP); cellulose acetate trimellitate; polymethacrylates (e.g., Eudragit® L series, Eudragit® S series, Eudragit® FS, Kollicoat® MAE 100P, Acryl-EZE® 93A, Acryl-EZE® MP); hydroxypropylmethylcellulose acetate succinate (HPMC AS; Aqoat®); sodium alginate; alginic acid, shellac and combinations thereof, and (c) an aqueous thickened flavored and sweetened liquid for suspending the particles.

11. The method of claim 9 wherein the liquid resin extended-release oral naltrexone formulation suspension is administered qAM (upon waking).
